(19) United States
(12) Patent Application Publication
(10) Pub. No.: US 2025/0288777 A1
(43) Pub. Date: Sep. 18, 2025

(54) PORT PATCHES

(71) Applicants: Carrie Shaltz Haslup, Glendale, CA (US); Philip Shaltz, Flint, MI (US)

(72) Inventors: Carrie Shaltz Haslup, Glendale, CA (US); Philip Shaltz, Flint, MI (US)

(21) Appl. No.: 19/083,127

(22) Filed: Mar. 18, 2025

Related U.S. Application Data
(60) Provisional application No. 63/566,575, filed on Mar. 18, 2024.

(51) Int. Cl. A61M 25/02; A41D 13/12

(52) U.S. Cl. CPC A61M 25/02; A41D 13/1236; A61M 2025/0206

(57) ABSTRACT

Disclosed are example embodiments of patches, garments, and methods for providing access to medical tubes and lines through clothing. A patch for enabling the passage of medical tubes or lines includes a panel made of a fabric material, a slit formed in the panel, and an adhesive backing for attaching the patch to clothing. A garment with an integrated access patch comprises a fabric panel and an access patch affixed to the fabric panel, wherein the slit of the access patch is configured to allow the passage of a medical tube or line through the fabric panel.

### CROSS-REFERENCE TO RELATED APPLICATIONS

The present application claims priority to U.S. Provisional Patent Application No. 63/566,575, filed Mar. 18, 2024, the disclosure of which is incorporated herein by reference in its entirety.

### TECHNICAL FIELD

This disclosure generally relates to the field of medical accessories, and more specifically and not by way of limitation, some embodiments are related to patches designed for providing access to medical tubes and lines through clothing.

### BACKGROUND

Children requiring medical tubes and lines, such as g-tubes, j-tubes, Peripherally Inserted Central Catheter (PICC) lines, and ports, often face challenges with clothing. Traditional solutions include cutting holes in clothing or using expensive specialty garments designed for medical equipment access. However, these methods may be unsightly, impractical, or costly.

Additionally, the emotional and psychological well-being of individuals with medical conditions is often overlooked when considering clothing modifications. The visible alterations to clothing, such as holes or visible medical devices, may lead to feelings of self-consciousness or embarrassment, particularly in social settings or among peers. This may harm the individual's confidence and overall quality of life.

Thus, a need exists for a solution that provides convenient and discreet access to medical tubes and lines through clothing without compromising comfort, aesthetics, or affordability. Some embodiments described herein address this need by offering versatile patches and garments with integrated access points that may be easily applied to various clothing items. This innovation may allow for a more seamless integration of medical equipment with everyday attire, enhancing the quality of life for individuals requiring medical access.

### SUMMARY

Some example embodiments relate to medical accessories, specifically to patches, garments, and methods designed to facilitate the passage of medical tubes and lines through clothing. These innovations may provide a practical solution for individuals who require access to medical tubes or lines, such as those used for feeding, medication delivery, or drainage, while maintaining comfort and convenience in their daily activities. Some embodiments encompass patches with adhesive backings that may be applied to clothing, garments with integrated access patches, and methods for applying and using these patches to ensure easy and discreet access to medical tubes and lines.

Disclosed are example embodiments of a patch for providing access to medical tubes and lines through clothing. The patch includes a panel made of a fabric material, a slit formed in the panel for the passage of a medical tube or line, and an adhesive backing for attaching the patch to clothing.

Disclosed are example embodiments of a method for providing access to medical tubes and lines through clothing. The method includes providing a patch, including a panel made of a fabric material, a slit formed in the panel, and an adhesive backing and applying the patch to a clothing item such that the slit aligns with a desired access point for a medical tube or line.

Disclosed are example embodiments of a garment with an integrated access patch. The garment includes a fabric panel configured to be worn by a user and an access patch affixed to the fabric panel. The access patch includes a fabric material, a slit formed in the fabric material, and an adhesive backing for attaching the access patch to the fabric panel, wherein the slit is configured to allow the passage of a medical tube or line through the fabric panel.

Disclosed are example embodiments of a method for providing access to medical tubes and lines through a garment. The method includes affixing an access patch to a fabric panel of the garment, aligning the slit of the access patch with a desired access point on the fabric panel, and configuring the slit to allow the passage of a medical tube or line through the fabric panel.

The features and advantages described in the specification are not all-inclusive. In particular, many additional features and advantages will be apparent to one of ordinary skill in the art in view of the drawings, specification, and claims. Moreover, it should be noted that the language used in the specification has been principally selected for readability and instructional purposes and may not have been selected to delineate or circumscribe the disclosed subject matter.

### BRIEF DESCRIPTION OF THE DRAWINGS

The foregoing summary, as well as the following detailed description, is better understood when read in conjunction with the accompanying drawings. The accompanying drawings, which are incorporated herein and form part of the specification, illustrate a plurality of embodiments and, together with the description, further serve to explain the principles involved and to enable a person skilled in the relevant art(s) to make and use the disclosed technologies.

FIG. 1 is a diagram illustrating a Port Patch in accordance with some embodiments of the systems and methods described herein.

FIG. 2 is a diagram illustrating the Port Patch of FIG. 1 installed on a garment, e.g., a “onesie,” in accordance with some embodiments of the systems and methods described herein.

FIG. 3 is a diagram illustrating the Port Patch of FIGS. 1-2 installed on a garment being worn by a baby, the Port Patch being used to allow a line to reach the baby under the garment in accordance with some embodiments of the systems and methods described herein.

FIG. 4 is a diagram illustrating the Port Patch of FIGS. 1-3 installed on a garment being worn by a baby, again, the Port Patch being used to allow a line to reach the baby under the garment in accordance with some embodiments of the systems and methods described herein.

FIG. 5 is a diagram illustrating the Port Patch of FIGS. 1-4 installed on a garment being worn by a baby, again, the Port Patch being used to allow a line to reach the baby under the garment in accordance with some embodiments of the systems and methods described herein.

FIG. 6 is a diagram illustrating the Port Patch of FIGS. 1-5 installed on a garment being worn by a baby, the Port Patch being used to allow a line to reach the baby under the garment in accordance with some embodiments of the systems and methods described herein.

FIG. 7 is a diagram illustrating the Port Patch of FIGS. 1-6 installed on a garment being worn by a baby, the diagram generally illustrates a side downward view of the garment in accordance with some embodiments of the systems and methods described herein.

FIG. 8 is a diagram illustrating example sizing for the Port Patch in accordance with some embodiments of the systems and methods described herein.

FIG. 9 is a flowchart illustrating an example method of applying a Port Patch to clothing in accordance with some embodiments of the systems and methods described herein.

FIG. 10 is a flowchart illustrating an example method of use of a garment with an integrated Port Patch in accordance with some embodiments of the systems and methods described herein.

The figures and the following description describe certain embodiments by way of illustration only. One skilled in the art will readily recognize from the following description that alternative embodiments of the structures and methods illustrated herein may be employed without departing from the principles described herein. Reference will now be made in detail to several embodiments, examples of which are illustrated in the accompanying figures. It is noted that wherever practicable similar or like reference numbers may be used in the figures to indicate similar or like functionality.

### DETAILED DESCRIPTION

The detailed description set forth below in connection with the appended drawings is intended as a description of configurations and is not intended to represent the only configurations in which the concepts described herein may be practiced. The detailed description includes specific details to provide a thorough understanding of various concepts. However, it will be apparent to those skilled in the art that these concepts may be practiced without these specific details.

The present disclosure provides an innovative solution to the aforementioned problems. Port Patches are patches that may be applied to any clothing, providing a neat, clean slit for medical tubes and lines to pass through. These Port Patchs offer customizable placement, ease of application, and versatility, making them a practical and stylish solution for individuals requiring medical access through their clothing.

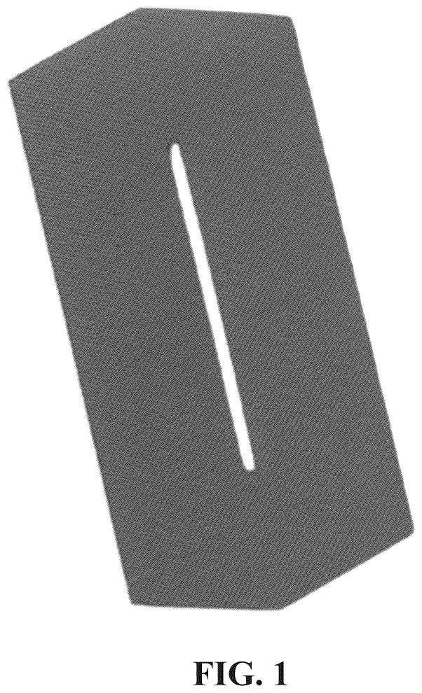

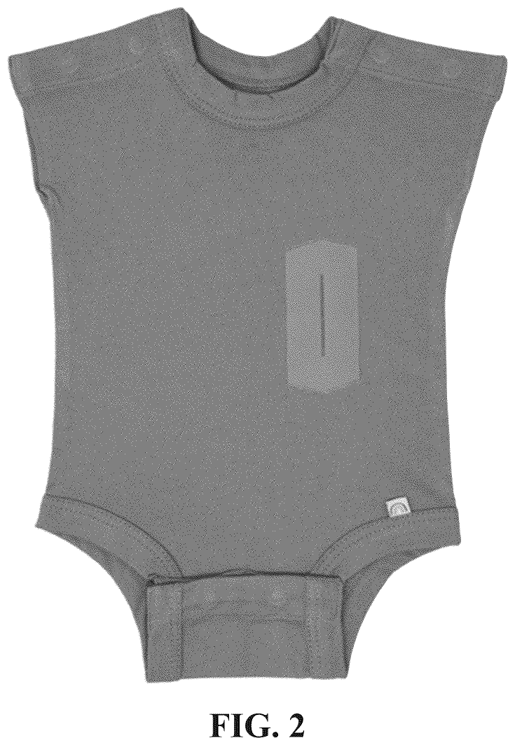

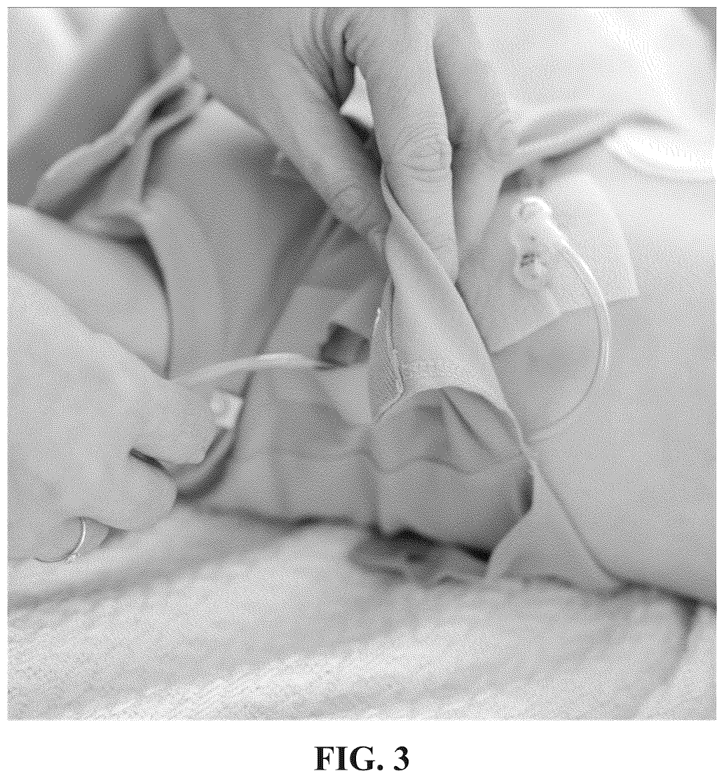

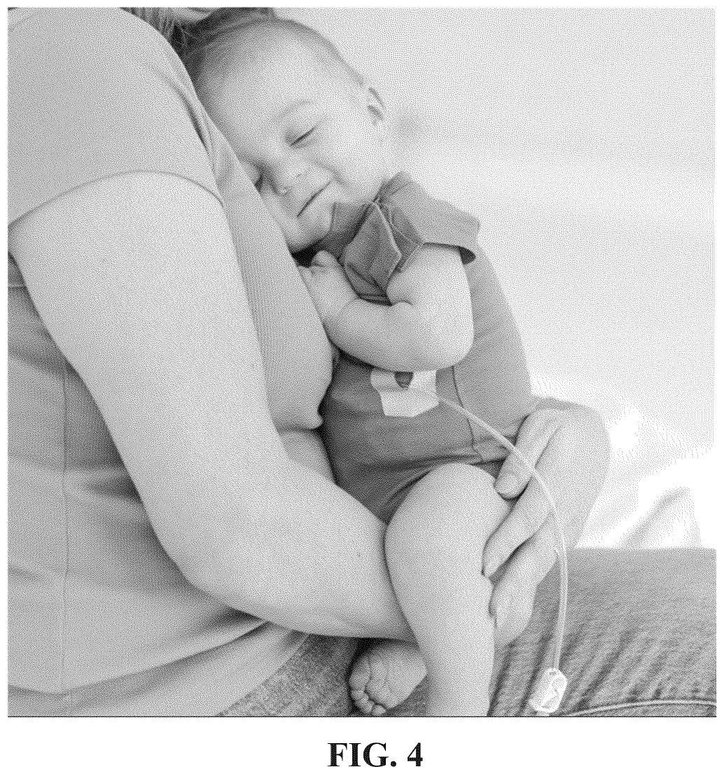

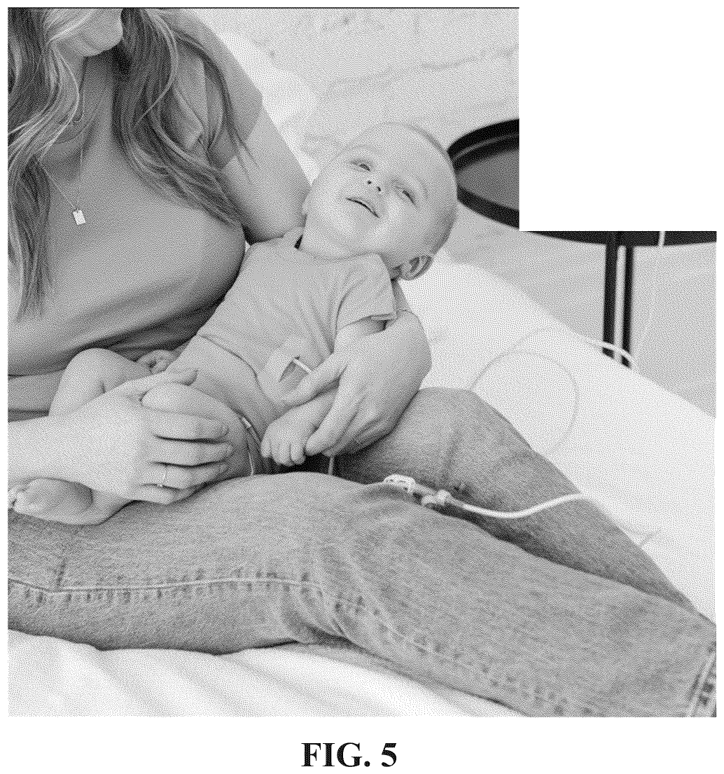

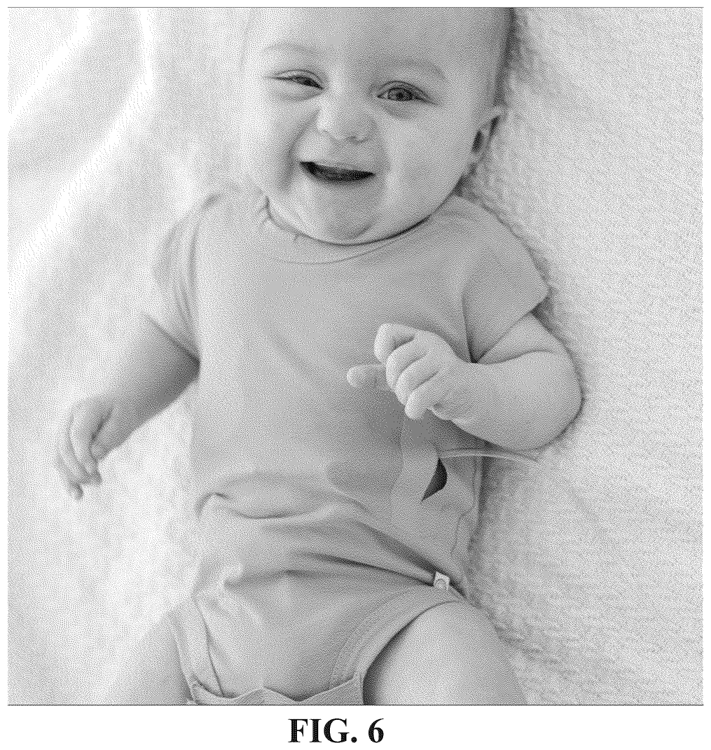

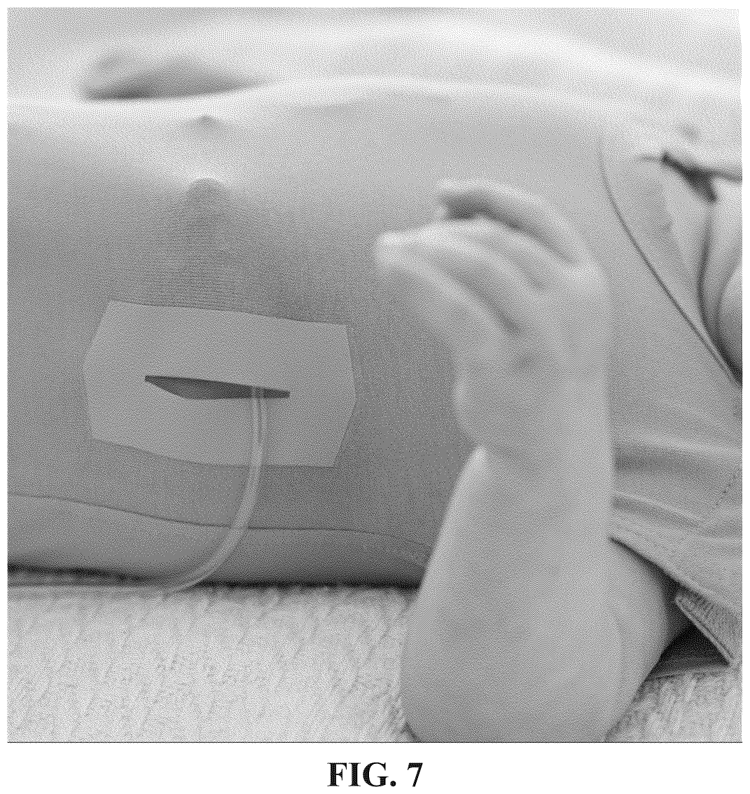

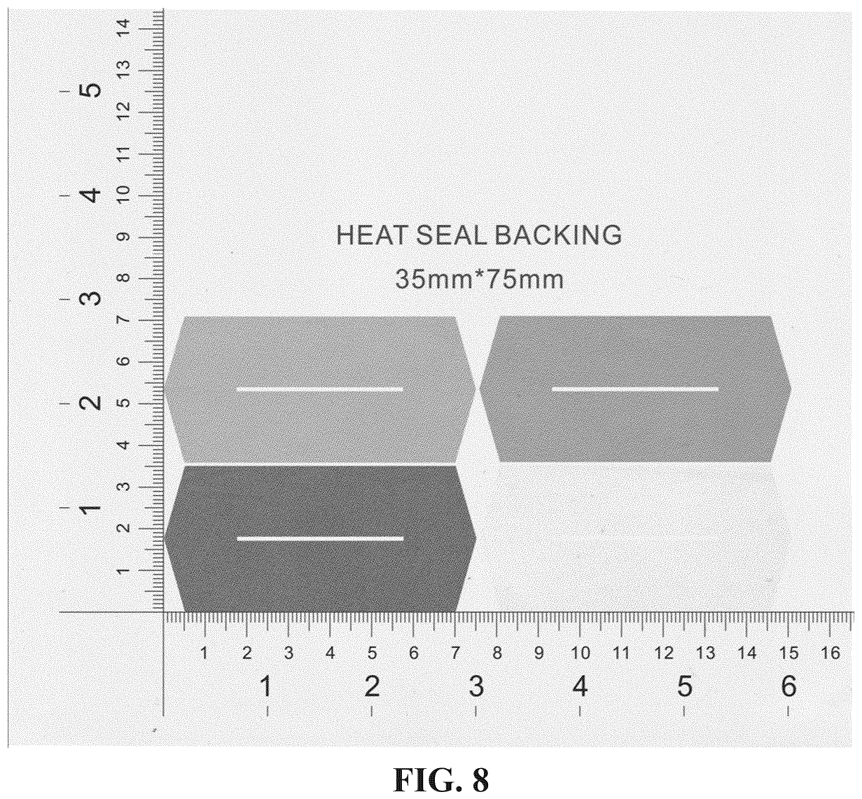

In an example embodiment, referring to FIGS. 1-8, the Port Patch (patch) is illustrated. In one example embodiment, the Port Patch may be made of a woven polyester rayon blend twill and may be laser-cut to specific dimensions, such as 35×75 mm, with a 1 mm slit cut into it. Other examples may have slits from 0.5 mm to 3 mm slits. Yet other embodiments may have slits anywhere from 0.25 mm to 4 mm or longer. Other example embodiments may have even shorter slits. The Port Patch may be backed with a commercial-grade heat-seal adhesive, allowing for easy application to any clothing. While one example embodiment of the Port Patch may be laser-cut to dimensions of 35×75 mm, it is to be understood that the Port Patch may be produced in various sizes to accommodate different clothing types and medical access needs. Additionally, embodiments may be laser cut or used any other appropriate method for cutting whatever material the particular embodiment may be made from. In an example embodiment, the thickness of the adhesive backing, mentioned as 0.15 mm, is exemplary, and the Port Patch may be constructed with adhesive layers of different thicknesses to achieve desired levels of adherence and flexibility.

In various embodiments, the Port Patch may be fabricated from a wide range of materials, each offering distinct properties and advantages. While one embodiment may utilize a woven polyester rayon blend twill, alternative materials such as cotton, spandex, breathable mesh fabrics, antimicrobial fabrics, or elastane blends may enhance comfort, hygiene, or stretchability. Additional materials for the Port Patch may include polyester-cotton blends for a balance between durability and comfort, denim for robust applications, and silk or satin for luxurious feel and formal wear. The choice of material may be tailored to the specific needs and preferences of the user, as well as the intended use and placement of the patch.

Beyond the adhesive blend of polyurethane (PU), polyamide (PA), polyethylene (PE), and ethylene-vinyl acetate (EVA), other adhesive options may include thermoplastic adhesive film, known for its strong bonding capabilities when heated and solidified upon cooling, and acrylic-based adhesives, recognized for their strong bond and resistance to heat and chemicals.

While one embodiment utilizes an iron-on method for attaching the Port Patch to clothing, it is understood that other attachment methods are possible and within the scope of the invention. These alternative methods may include sew-in patches for a permanent and secure attachment, Velcro patches for easy attachment and removal, button-on patches using buttons for attachment, snap-on patches with snap buttons for a more secure fit, and adhesive fabric tape for temporary attachment without the need for sewing or ironing. It should be noted that these attachment methods are examples, and other methods may be employed without deviating from the scope of the invention.

In light of the various materials and attachment methods available, the design and application of Port Patches may be customized to suit the specific needs and preferences of the user. For instance, a user with sensitive skin may opt for a Port Patch made of hypoallergenic materials with a medical-grade silicone adhesive, while an athlete may prefer a stretchable and breathable Port Patch with a snap-on attachment for easy removal and reattachment.

The adaptability of Port Patches extends to their shape and size. While the standard shape may be rectangular or oval, custom shapes such as stars, hearts, or even logos may be created to add a personal touch or branding element to the patch. The size of the Port Patch may also be tailored to the size of the medical device or the area of the clothing where the Port Patch will be applied.

Additional features may be incorporated to enhance the functionality of Port Patches further. For example, patches with built-in pockets or pouches may provide storage space for medical devices, while patches with integrated sensors may monitor the condition of the skin or the status of the medical device. Reflective or glow-in-the-dark materials may be used to increase visibility and safety in low-light conditions, e.g., in an example with a built-in pocket, which may allow easier access to medical devices within the pocket, in low-light conditions, for example.

The versatility of Port Patches also allows for their use in various settings beyond medical applications. For example, Port Patches may be used in outdoor gear to access hydration tubes or with pet clothing for veterinary devices. The vast potential applications demonstrate this innovative solution's broad appeal and utility.

In addition to their practical applications, Port Patches may also serve aesthetic purposes. The patches may be designed with decorative elements, such as embroidery, sequins, or prints, to make them a fashionable accessory. This may help reduce the stigma associated with medical devices and make users feel more confident and stylish while wearing them. In other example embodiments, the opposite may be true. Rather than being designed to be decorative, the Port Patch may be designed to blend in with the rest of the clothing and make the Port Patch less noticeable.

Port Patches may also be integrated into smart clothing systems. With the advancement of wearable technology, patches may be configured to allow for connectivity features, such as Bluetooth or RFID, to communicate with smartphones or medical monitoring devices. For example, the Port Patch may allow for cabling for various types of devices to be routed from above the clothing to underneath the clothing. The Port Patch may enable real-time tracking of medication delivery, fluid intake, or other vital health parameters.

The adhesive backing of the Port Patch may be used to secure attachment to clothing. While a blend of polyurethane (PU), polyamide (PA), polyethylene (PE), and ethylene-vinyl acetate (EVA) may commonly be used, alternative embodiments may utilize medical-grade silicone adhesives for sensitive skin, pressure-sensitive adhesives for easy repositioning, or water-resistant adhesives for durability in wet conditions. The adhesive may be applied in layers of varying thicknesses to achieve desired levels of adherence and flexibility. In addition to examples using a woven polyester rayon blend twill, other examples of the Port Patch may be fabricated from other materials such as breathable mesh fabrics for enhanced comfort, antimicrobial fabrics for improved hygiene, or stretchable fabrics like elastane blends to accommodate movement and different body shapes.

In some embodiments, Port Patches may incorporate advanced fabric technology to enhance comfort, durability, and adaptability to different environmental conditions. For example, moisture-wicking fabrics may be utilized to manage perspiration and prevent skin irritation, particularly for individuals wearing medical devices for extended periods. Additionally, thermoregulating materials, such as phase-change textiles, may be integrated into the Port Patch to help maintain an optimal skin temperature and prevent overheating. For individuals with sensitive skin, hypoallergenic coatings or medical-grade silicone adhesives may be employed to reduce the risk of irritation or allergic reactions. Moreover, in embodiments designed for extended use, antimicrobial treatments may be applied to prevent bacterial growth and ensure a hygienic interface between the Port Patch and the user's clothing.

Further, the Port Patch may incorporate features that improve user experience and ease of application. Some embodiments may include perforated adhesive layers that allow users to remove protective backing in sections, providing more control during installation. This feature may be particularly beneficial for caregivers applying patches to young children or individuals with limited dexterity. In another embodiment, the Port Patch may include color-coded adhesive indicators that assist with alignment, ensuring precise placement over medical access points. These improvements may contribute to a more user-friendly and effective solution for individuals who require discreet and reliable access to medical tubing through clothing.

Additionally, Port Patches may be designed with modular attachment systems that allow users to customize placement or replace worn patches without altering the garment. For example, some configurations may use removable magnetic fasteners or reusable hook-and-loop mechanisms to allow for easy repositioning and reuse. These designs may be particularly advantageous for users who need frequent access to different medical lines or who prefer to alternate between clothing options while maintaining consistent access points.

In an example embodiment, the dimensions and proportions of the patch, as well as the slit, may be adjusted to optimize compatibility with various garment styles and sizes, ensuring a versatile and universally applicable solution.

The Port Patch may be produced in various shapes, including but not limited to circular, oval, or custom shapes, to fit different clothing styles and better provide for access needs. The Port Patch size may also be adjustable, ranging from small patches for discreet access to larger patches for accommodating multiple tubes or lines.

In an example embodiment, the slit formed in the patch, although described as being 1 mm in length in one embodiment, may be varied in size to suit the specific requirements of the medical tubes or lines intended to pass through it. The adhesive may provide a strong bond that may withstand regular wear and washing. The Port Patch may be applied to various clothing items, such as onesies, shirts, pants, blankets, and bibs.

While one embodiment utilizes a polyester rayon blend twill, the Port Patch may be made from various materials, including but not limited to cotton, nylon, spandex blends, or any other appropriate material. The size and shape of the patch, as well as the size of the slit, may be varied to accommodate different needs and preferences.

In an example embodiment, the comfort and convenience of users may be considered in the design of Port Patches. By offering seamless integration with everyday clothing, these patches may alleviate the need for specialized medical garments, allowing users to maintain their style while accommodating medical issues. The Port Patches may be designed to be soft and flexible, ensuring that the Port Patches do not irritate the skin or cause discomfort during movement. This user-centric approach may ensure that individuals can do their daily activities with minimal disruption, enhancing their overall quality of life.

In an example embodiment, Port Patches may be crafted with durability and capable of withstanding the rigors of daily wear and frequent laundering. The materials used may be selected for their resilience and ability to retain their adhesive properties and structural integrity over time. Users may be advised to follow specific care instructions, which include washing the garments inside out on a gentle cycle and avoiding direct heat when ironing to ensure the longevity of the patches,. By adhering to these guidelines, users may enjoy the benefits of the patches for an extended period.

In an example embodiment, recognizing the importance of personal expression, Port Patches may be available in various colors, patterns, and sizes to suit diverse tastes and preferences. This level of customization may allow users to choose patches that complement their clothing, making the medical accessory a stylish addition to their wardrobe. Users may find options that align with their aesthetic preferences, whether seeking a subtle, discreet Port Patch or a bold, decorative accent.

In further embodiments, the garment with an integrated access Port Patch may be designed as specialized medical wear, such as hospital gowns or surgical scrubs, providing easy access for medical procedures while maintaining patient comfort and dignity. Additionally, sports and activewear with integrated access patches may be developed to accommodate athletes and individuals with active lifestyles who require medical access during physical activities. Elder care applications may also benefit from the Port Patch, particularly for individuals requiring long-term IV therapy, feeding tubes, or catheter access. The discreet and secure design may help preserve patient dignity while reducing the need for specialized garments. Similarly, post-surgical garments incorporating Port Patches may facilitate drainage tube management following procedures such as mastectomies, abdominal surgeries, or wound care, allowing for easier access while promoting healing and reducing irritation.

Customization options may extend beyond colors and patterns to include personalized text or symbols, allowing users to add medical information or personal identifiers to the patch. This feature may be particularly useful in emergencies or distinguishing between multiple users' garments.

In an example embodiment, the safety and hygiene of users may be considerations in the development of Port Patches. The materials used may be carefully selected for their hypoallergenic properties, minimizing the risk of skin irritation or allergic reactions. Additionally, some patches may feature antimicrobial coatings to inhibit the growth of bacteria and maintain a hygienic interface between the medical device and the user's skin. These features may underscore the commitment to providing a safe and clean solution for managing medical tubes and lines.

In some embodiments, the Port Patch may feature enhancements such as waterproof coatings to protect against moisture and liquid spills or reflective elements for increased visibility and safety in low-light conditions. These features may further extend the functionality and versatility of the Port Patch, making the Port Patch suitable for a broader range of applications and environments.

In an example embodiment, following the correct application process for Port Patches may be necessary to ensure optimal performance. Users may start by identifying the clothing location where the Port Patch is needed. The area should be clean and flat, with the garment on a heat- resistant surface. The Port Patch may then be positioned with the adhesive side facing down, and heat may be applied using an iron or a heat press, following any recommended temperature and duration settings for the particular adhesive. Once affixed, the Port Patch may be allowed to cool before the garment is worn. This straightforward application process may ensure a strong bond and proper alignment for practical use.

In the method of applying the Port Patch to a clothing item, it may be necessary to ensure that the slit formed in the panel made of the fabric material aligns with the desired access point for a medical tube or line. This alignment allows the medical tube or line to be inserted and accessed through the clothing effectively.

In certain embodiments, the Port Patch may be integrated with wearable technology, such as sensors or monitoring devices, to provide real-time health data or alerts. This integration may offer added convenience and peace of mind for users who require health monitoring, making the Port Patch a multifunctional tool in healthcare management.

The adhesive backing of the Port Patch may be designed to provide a strong, durable bond to clothing while being gentle on the skin. The Port Patch may be formulated to withstand multiple wash cycles without losing the Port Patch's adhesive properties. The adhesive may also withstand a range of temperatures, making the adhesive suitable for use in various climates and conditions.

In some embodiments, the manufacturing process of the Port Patch may involve several steps, including material selection, laser cutting to precise dimensions, and application of the adhesive backing. In some embodiments, the Port Patch may be utilized in sports medicine to provide athletes with a convenient way to manage medical devices or medication delivery while engaged in physical activities. The Port Patch May also be used in post-surgical care to provide easy access to surgical sites for monitoring and treatment.

The use of Port Patches offers several benefits over traditional methods, including reduced discomfort from friction between medical devices and clothing, easier access for medical treatment, and the ability to maintain a normal appearance without the need for specialized clothing. While the Port Patch provides a convenient solution for accessing medical tubes and lines, it is important to consider factors such as skin sensitivity and the specific medical requirements of the user.

In some embodiments, the Port Patch may include a multi-layer construction to provide enhanced durability and flexibility. For example, the patch may incorporate a breathable outer fabric layer, a moisture-wicking intermediate layer, and a reinforced adhesive backing. The outer fabric layer may be composed of materials such as microporous polyester, breathable cotton blends, or elastane-infused fabrics to provide comfort while maintaining structural integrity. The moisture-wicking layer may help prevent irritation and skin discomfort by directing perspiration away from the adhesive backing.

Additionally, in alternative embodiments, the Port Patch may include a waterproof barrier layer, preventing fluid exposure or accidental spills from compromising the adhesion of the patch to clothing. This can be particularly useful in pediatric applications or for users requiring extended wear periods.

Some embodiments of the Port Patch may utilize biodegradable materials to provide an eco-friendly alternative for disposable patches. These patches may be constructed using plant-based fibers, biodegradable thermoplastics, or compostable adhesive layers, making them suitable for short-term medical use without contributing to long-term environmental waste.

In some configurations, the slit formed in the Port Patch may include reinforced stitching, polymer edge-coating, or self-sealing technology to provide enhanced durability and prevent fabric fraying over time. For example, heat-sealed polymer linings or flexible silicone-based edges may be applied along the slit to reduce wear and tear while maintaining a secure opening for medical tubing.

Certain embodiments may feature adjustable slit designs that can expand or contract depending on the thickness of the medical tube. This can be achieved using elasticized materials around the slit opening or by integrating a flexible membrane that adapts to different tube diameters. Such a configuration may minimize unnecessary exposure while maintaining a snug fit around medical lines.

While many embodiments use an adhesive backing, the Port Patch may also be mechanically secured using various fastening mechanisms. Examples of alternative attachment methods include:

Magnetic closures: The Port Patch may integrate a thin magnetic strip embedded within the fabric to allow for temporary and adjustable placement on metallic garment surfaces.

Hook-and-loop fasteners (Velcro®): The patch may include a reusable fastener system, allowing users to attach and reposition the patch multiple times.

Snap-button closures: Some configurations may feature integrated snap fasteners along the edges of the patch, securing it to pre-designated sections of a modified garment.

These alternative fastening methods allow non-permanent attachment of the Port Patch, enabling reuse across multiple garments.

In some embodiments, Port Patches may integrate wearable sensors or electronic components to enhance their functionality. For example, a sensor-equipped Port Patch may:

Monitor temperature at the tube exit point to help detect infections or abnormal skin reactions. These smart patches may be particularly useful for hospital environments, pediatric care, and remote patient monitoring, providing real-time alerts to caregivers if a medical line becomes dislodged or obstructed.

The Port Patch may be manufactured using various techniques to accommodate different applications and cost considerations. Some example manufacturing processes include:

Laser cutting: Precision laser-cutting techniques may be used to ensure uniform slit dimensions and sealed fabric edges, preventing fraying.

Ultrasonic welding: In certain embodiments, ultrasonic bonding methods may be employed to fuse multi-layered fabrics without the use of adhesives or stitching.

3D printing and additive manufacturing: For specialized applications, 3D-printed polymer-based Port Patches may be produced, allowing customized geometries and integrated reinforcement structures.

The Port Patch may also be customizable for individual patient needs, branding, or personalization. Some embodiments may allow users to select:

Custom patch colors and patterns for aesthetic preference.

Embroidery or printed labels for medical information or emergency contact details.

Size variations, from small, discreet patches to larger patches designed for multi-line access.

While the primary focus of Port Patches is medical tube access, the design may be adapted for alternative industries. Some possible non-medical applications include:

Sports and fitness wear: Port Patches may be used in athletic clothing to provide convenient access to hydration tubes for long-distance runners and endurance athletes.

Wearable technology integration: In certain embodiments, the patches may serve as pass-through access points for wearable electronics, such as wired audio systems or biometric monitoring devices.

Pet and veterinary applications: Port Patches may be used in animal harnesses or post-surgical garments for veterinary applications, allowing medical tubing or sensors to pass through pet clothing.

In some embodiments, the access patch may include a moisture-resistant or antimicrobial coating to enhance hygiene and durability. The moisture-resistant coating may be applied as a thin polymer layer, such as polyurethane, polyethylene, or silicone, to prevent liquid penetration and ensure the patch remains securely adhered to the garment. This can be particularly beneficial for individuals who may experience fluid exposure or prolonged moisture contact due to medical conditions.

Alternatively, the antimicrobial coating may be applied to the patch to inhibit bacterial or fungal growth, reducing the risk of infections or skin irritation. Suitable antimicrobial treatments may include silver ion coatings, copper-infused fabrics, or medical-grade antimicrobial agents such as quaternary ammonium compounds (QACs), triclosan, or zinc-based treatments. In one embodiment, the antimicrobial coating is integrated into the fabric material itself through a dip-coating process, spray application, or heat-sealed infusion.

The choice of moisture-resistant or antimicrobial coatings may depend on the intended application of the Port Patch. For example, pediatric or long-term care patients may benefit from antimicrobial protection, whereas athletes or individuals with excessive perspiration may prefer moisture-resistant properties to maintain patch integrity over time.

In some embodiments, the slit formed in the access patch may incorporate a reinforced structure designed to provide additional flexibility and durability while accommodating various tubing diameters. The reinforcement may be achieved through:

Flexible polymer membranes, such as silicone or thermoplastic elastomers (TPEs), which maintain a tight seal around inserted medical tubes to prevent unintentional movement while ensuring ease of insertion and removal.

Coated edges, where the fabric surrounding the slit is heat-sealed or reinforced with polyurethane, polyamide, or synthetic rubber linings, reducing fraying or wear over extended use.

In an example embodiment, the slit may be lined with an elastic fabric blend, such as a combination of spandex, Lycra®, or stretchable polyester, to create a self-adjusting opening that conforms to different tube sizes. This design helps ensure that the slit remains secure without excessively stretching or tearing, providing long-term durability and user comfort.

Another variation may involve a dual-layer membrane structure, where an outer elastic layer provides resistance to movement, while an inner soft-sealing membrane (e.g., silicone gel or foam padding) provides cushioning against friction and pressure on the skin.

The use of reinforced and flexible slit designs allows the Port Patch to be customized for different medical tubing applications, including feeding tubes, PICC lines, drainage catheters, and insulin pump tubing.

While some embodiments of the Port Patch feature a permanent adhesive backing, other embodiments may utilize removable fastening mechanisms, allowing for repositioning or reuse. These fastening mechanisms may include:

Hook-and-loop fasteners (Velcro®): The patch may feature a hook-and-loop system attached to both the garment and the patch, allowing users to remove and reposition the patch as needed.

Magnetic strips: The patch may include thin embedded magnets that align with a corresponding magnetic base on the garment, providing secure attachment while allowing easy removal.

Snap-button closures: The patch may feature snap-button mechanisms, where the patch can be securely snapped onto pre-installed button studs in the garment.

Adhesive strips with peel-off layers: Instead of a single-use adhesive, the patch may include multi-use adhesive strips that retain adhesion after multiple applications.

In an example embodiment, the hook-and-loop fastener version of the Port Patch may include a sewn-in loop fabric on the patch itself and a hook strip adhered to the garment, allowing for easy replacement or repositioning of the patch without damaging the clothing. This configuration may be beneficial for children who outgrow clothing or individuals who require frequent garment changes.

The magnetic strip variation may include flexible rare-earth magnets embedded within the patch fabric, providing a secure, non-permanent attachment option. This is particularly useful for delicate fabrics where adhesive or sewing methods may not be ideal.

These alternative attachment methods provide users with customization options based on their specific needs, offering a balance between secure medical access and garment versatility.

In some embodiments, the access patch may be heat-sealed or incorporate a thermally resistant layer to enhance durability and prevent fraying of the fabric surrounding the slit. This may be particularly useful for high-wear applications, such as for infants, active users, or long-term medical device wearers, where frequent movement and washing may lead to fabric degradation.

One approach for thermal resistance includes applying a polymer-based heat-seal treatment to the fabric edges. Suitable materials may include:

Polyurethane (PU) coatings, which provide a flexible yet strong protective barrier.

Silicone-based reinforcements, offering soft, flexible, and heat-resistant properties.

High-temperature synthetic fibers, such as aramid blends (e.g., Kevlar®) or Nomex®, for increased resilience.

In an example embodiment, the edges of the slit may be laser-cut and heat-sealed simultaneously, ensuring precision while preventing loose fibers. This method enhances the structural integrity of the Port Patch while maintaining aesthetic and functional quality over time.

Alternative embodiments may utilize laminated or dual-layer construction, where a thin thermoplastic polyurethane (TPU) layer is bonded to the underside of the fabric, providing added reinforcement without sacrificing flexibility.

The inclusion of a thermal-resistant or heat-sealed layer allows the Port Patch to withstand multiple wash cycles, reduce wear and tear, and maintain a consistent slit opening size for extended use.

Heat-sealing may generally be applied to all fabric types disclosed, ensuring enablement across embodiments.

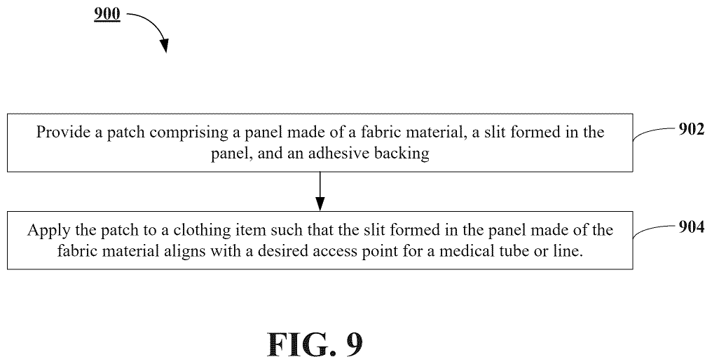

FIG. 9 is a flowchart illustrating a method 900 of applying a Port Patch to clothing. Method 900 includes providing a Port Patch comprising a panel made of a fabric material, a slit formed in the panel, and an adhesive backing (902). Methods 900 also includes aligning the Port Patch with the desired access point on the clothing item (904). Additionally, method 900 includes applying heat to the Port Patch to activate the adhesive backing to affix the Port Patch to the clothing (906). Other embodiments may use different methods to apply the Port Patch.

Method 900 includes providing a Port Patch comprising a panel made of a fabric material, a slit formed in the panel, and an adhesive backing (902). A Port Patch may include a panel of fabric material, such as a woven polyester rayon blend twill. The Port Patch may also have a slit formed in the panel, which may be designed to allow a medical tube or line to pass through the Port Patch. The Port Patch may have an adhesive backing, which may be a blend of polyurethane, polyamide, polyethylene, and ethylene/vinyl acetate.

Method 900 also includes aligning the Port Patch with the desired access point on the clothing item (904). The Port Patch may be aligned with the desired access point on the clothing item. This step may ensure that the slit in the Port Patch is positioned correctly to allow easy access to the medical tube or line.

Additionally, method 900 includes applying heat to the Port Patch to activate the adhesive backing to affix the Port Patch to the clothing (906). Heat may be applied to the Port Patch to activate the adhesive backing, which may be done using an iron, heat gun, heat press, or some other heat source. The heat may cause the adhesive to bond with the clothing, securely affixing the patch.

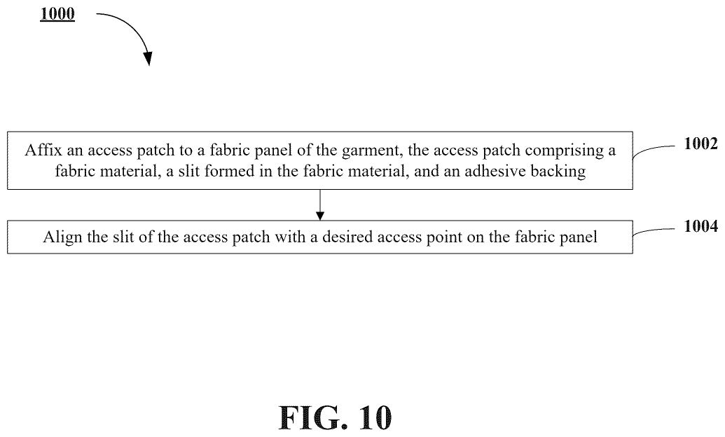

FIG. 10 is a flowchart illustrating an example of use of a garment with an integrated Port Patch. The method 1000 may include affixing an access Port Patch to a fabric panel of the garment, the access Port Patch comprising a fabric material, a slit formed in the fabric material, and an adhesive backing (1002). The method 1000 may also include aligning the slit of the access Port Patch with a desired access point on the fabric panel (1004). Additionally, the method may include wearing the garment, allowing the slit in the access Port Patch to provide access for a medical tube or line through the fabric panel (1006).

Method 1000 may include affixing an access Port Patch to a fabric panel of the garment, the access Port Patch comprising a fabric material, a slit formed in the fabric material, and an adhesive backing (1002). In this step, the access Port Patch is attached to the fabric panel of the garment. The Port Patch may include a fabric material with a slit and an adhesive backing. The adhesive backing may ensure the Port Patch stays securely on the garment.

Method 1000 may also include aligning the slit of the access Port Patch with a desired access point on the fabric panel (1004). The slit in the access Port Patch is aligned with the desired access point on the fabric panel. This alignment may help ensure that the medical tube or line may pass through the garment at the appropriate location.

Additionally, method 1000 may include wearing the garment, allowing the slit in the access Port Patch to provide access for a medical tube or line through the fabric panel (1006). The user wears the garment with the integrated access patch. The slit in the Port Patch provides a convenient and discreet access point for medical tubes or lines, allowing for easy and comfortable use.

While the embodiments illustrated in the figures and described herein may show the Port Patch applied to a baby's garment, e.g., a onesie, it is to be understood that this is merely an example of one potential application. The Port Patch is designed to be versatile and may be applied to clothing for individuals of all ages, including not just babies but toddlers, children, teens, and adults. Furthermore, the concept of the Port Patch may be extended to include applications for animal clothing, providing a solution for pets or other animals that require medical tubes or lines. This flexibility in application underscores the wide-ranging utility of the Port Patch in various contexts.

One or more elements or aspects or steps, or any portion(s) thereof, from one or more of any of the systems and methods described herein may be combined with one or more elements or aspects or steps, or any portion(s) thereof, from one or more of any of the other systems and methods described herein and combinations thereof, to form one or more additional implementations and/or claims of the present disclosure.

One or more of the components, steps, features, and/or functions illustrated in the figures may be rearranged and/or combined into a single component, block, feature or function or embodied in several components, steps, or functions. Additional elements, components, steps, and/or functions may also be added without departing from the disclosure. The apparatus, devices, and/or components illustrated in the Figures may be configured to perform one or more of the methods, features, or steps described in the Figures. The algorithms described herein may also be efficiently implemented in software and/or embedded in hardware.

Reference in the specification to “one embodiment” or “an embodiment” means that a particular feature, structure, or characteristic described in connection with the embodiment is included in at least one embodiment of the invention. The appearances of the phrase “in one embodiment” in various places in the specification are not necessarily all referring to the same embodiment.

The figures and the following description describe certain embodiments by way of illustration only. One skilled in the art will readily recognize from the following description that alternative embodiments of the structures and methods illustrated herein may be employed without departing from the principles described herein. Reference will now be made in detail to several embodiments, examples of which are illustrated in the accompanying figures. It is noted that wherever practicable similar or like reference numbers may be used in the figures to indicate similar or like functionality.

The foregoing description of the embodiments of the present invention has been presented for the purposes of illustration and description. It is not intended to be exhaustive or to limit the present invention to the precise form disclosed. Many modifications and variations are possible in light of the above teaching. It is intended that the scope of the present invention be limited not by this detailed description, but rather by the claims of this application. As will be understood by those familiar with the art, the present invention may be embodied in other specific forms without departing from the spirit or essential characteristics thereof. Likewise, the particular naming and division of the modules, routines, features, attributes, methodologies and other aspects are not mandatory or significant, and the mechanisms that implement the present invention or its features may have different names, divisions and/or formats.

It is understood that the specific order or hierarchy of blocks in the processes/flowcharts disclosed is an illustration of example approaches. Based on design preferences, it is understood that the specific order or hierarchy of blocks in the processes/flowcharts may be rearranged. Further, some blocks may be combined or omitted. The accompanying method claims present elements of the various blocks in a sample order and are not meant to be limited to the specific order or hierarchy presented.

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” The word “exemplary” is used herein to mean “serving as an example, instance, or illustration.” Any aspect described herein as “exemplary” is not necessarily to be construed as preferred or advantageous over other aspects. Unless specifically stated otherwise, the term “some” refers to one or more. Combinations such as “at least one of A, B, or C,” “one or more of A, B, or C,” “at least one of A, B, and C,” “one or more of A, B, and C,” and “A, B, C, or any combination thereof” include any combination of A, B, and/or C, and may include multiples of A, multiples of B, or multiples of C. Specifically, combinations such as “at least one of A, B, or C,” “one or more of A, B, or C,” “at least one of A, B, and C,” “one or more of A, B, and C,” and “A, B, C, or any combination thereof” may be A only, B only, C only, A and B, A and C, B and C, or A and B and C, where any such combinations may contain one or more member or members of A, B, or C. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public, regardless of whether such disclosure is explicitly recited in the claims. The words “module,” “mechanism,” “element,” “device,” and the like may not be a substitute for the word “means.” As such, no claim element is to be construed as a means plus function unless the element is expressly recited using the phrase “means for.”

What is claimed is:

1. A patch for providing access to medical tubes and lines through clothing, comprising:
a panel made of a fabric material;
a slit formed in the panel, through which a medical tube or line can pass; and
an adhesive backing for attaching the patch to clothing.

2. The patch of claim 1, wherein the fabric material is a woven polyester rayon blend twill.

3. The patch of claim 1, wherein the adhesive backing comprises a blend of polyurethane, polyamide, polyethylene, and ethylene/vinyl acetate.

4. The patch of claim 1, wherein the panel is laser-cut to specific dimensions.

5. The patch of claim 1, wherein the slit is 1 mm in length.

6. A method for providing access to medical tubes and lines through clothing, comprising:
providing a patch comprising a panel made of a fabric material, a slit formed in the panel, and an adhesive backing; and
applying the patch to a clothing item such that the slit formed in the panel made of the fabric material, wherein the fabric material aligns with a desired access point for a medical tube or line.

7. The method of claim 6, wherein the fabric material is a woven polyester rayon blend twill.

8. The method of claim 6, wherein the adhesive backing comprises a blend of polyurethane, polyamide, polyethylene, and ethylene/vinyl acetate.

9. The method of claim 6, wherein the panel is laser-cut to specific dimensions.

10. The method of claim 6, wherein the slit is 1 mm in length.

11. A garment with an integrated access patch, comprising:
a fabric panel configured to be worn by a user;
an access patch affixed to the fabric panel, the access patch comprising a fabric material, a slit formed in the fabric material, and an adhesive backing for attaching the access patch to the fabric panel; and
wherein the slit is configured to allow passage of a medical tube or line through the fabric panel.

12. The garment of claim 11, wherein the fabric material of the access patch is a woven polyester rayon blend twill.

13. The garment of claim 11, wherein the adhesive backing of the access patch comprises a blend of polyurethane, polyamide, polyethylene, and ethylene/vinyl acetate.

14. The garment of claim 11, wherein the access patch is laser-cut to specific dimensions.

15. The garment of claim 11, wherein the slit in the access patch is 1 mm in length.

16. The garment of claim 11, wherein the fabric panel is configured as a onesie, shirt, pant, or blanket.

17. The garment of claim 11, wherein the access patch further comprises a moisture-resistant or antimicrobial coating to prevent bacterial buildup and enhance hygiene.

18. The garment of claim 11, wherein the access patch includes a reinforced slit with a flexible membrane or elasticized material, allowing for a secure yet adaptable opening that accommodates various tube diameters while minimizing exposure.

19. The garment of claim 11, wherein the access patch is attached using a removable fastening mechanism, selected from the group consisting of hook-and-loop fasteners, magnetic strips, snap buttons, or adhesive strips, allowing for repositioning or reuse across multiple garments.

20. The garment of claim 11, wherein the access patch comprises a heat-sealed or thermally resistant layer to enhance durability and prevent fraying of the fabric surrounding the slit.
